(19) United States
(12) Patent Application Publication
(10) Pub. No.: US 2015/0065319 A1
(43) Pub. Date: Mar. 5, 2015

(54) PROCESS FOR EXTENDING AND EXERCISING ONE'S BACK

(71) Applicant: ROBERT ALLAN WALD, BURBANK, CA (US)

(72) Inventor: ROBERT ALLAN WALD, BURBANK, CA (US)

(21) Appl. No.: 14/017,130

(22) Filed: Sep. 3, 2013

(51) Int. Cl. A61F 5/042

(52) U.S. Cl. CPC A61F 5/042
USPC 482/132

(57) ABSTRACT

A process for extending and stretching using a rocking back stretcher permitting a desired stretch. The process involves the following steps. First is lying on a cushion connected to a seat plate. The seat plate is connected to a right corner brace and a left corner brace. The right corner brace is connected to a right leg. The left corner brace is connected to a left leg. The right leg and the left leg are connected to a front cross bar and a rear cross bar. Next is rocking forward until the desired stretch is achieved or the front cross bar makes contact with ground or rocking backward until the desired stretch is achieved or the rear cross bar makes contact with the ground. After that is holding the desired stretch once it is achieved.

### RELATED APPLICATION

This application claims priority to provisional patent application U.S. Ser. No. 61/701,796 filed on Sep. 17, 2012, the entire contents of which is herein incorporated by reference.

### BACKGROUND

The embodiments herein relate generally to exercise equipment.

Prior to embodiments of the disclosed process, there were no back stretching devices presently on the market which are capable of allowing the user to make full use of one's own body weight and gravity to achieve doctor recommended, fully arched upper body stretches, with the added rocking capability. Further there was no device that further permitted a user to sit in a way that promotes much better posture and, therefore, less back pain. Additionally, no product presently available that specifically addresses the need for better circulation in the lower extremities while sitting. The prior art includes U.S. Design Pat. D373425 issued to Palmisano.

Palmisano had a good general idea which failed in the details. The cross supports in Palmisano were insufficient to support the weight of a heavy user. This is probably because obesity was not much of a design consideration in 1990, but it is prevalent today. Embodiments of the present invention use reinforced screw sockets to provide additional strength. Palmisano was not concerned with the device slipping. Embodiments of the present invention solve this problem with a friction increasing liner. Finally, Palmisano chose a cushion of an inadequate geometry when the device is used as both a chair and as a back stretching device.

Arched stretching, especially in a controlled way, has been accepted by spine surgeons as superior to linear stretching and over unstable balance balls. We have done the research to substantiate these claims. For example, linear stretching, such as with an inversion table, is not recommended by orthopedists for post-surgery spine therapy, whereas arched stretches are commonly recommended. Balance balls are too strenuous for many people who suffer from back pain, and they are inherently dangerous, especially for older individuals or for those with balance issues or weak muscles.

### SUMMARY

A process for extending and stretching using a rocking back stretcher permitting a desired stretch. The process involves the following steps. First is lying on a cushion connected to a seat plate. The seat plate is connected to a right corner brace and a left corner brace. The right corner brace is connected to a right leg. The left corner brace is connected to a left leg. The right leg and the left leg are connected to a front cross bar and a rear cross bar. Next is rocking forward until the desired stretch is achieved or the front cross bar makes contact with ground or rocking backward until the desired stretch is achieved or the rear cross bar makes contact with the ground. After that is holding the desired stretch once it is achieved.

In some embodiments, the right leg and the left leg are connected to the front cross bar and the rear cross bar with threaded cylinders to accommodate more weight. The cushion comprises medium density foam, polyester batting and an anti-bacterial vinyl cover to provide comfort. The cushion is larger than the seat plate.

A rocking back stretcher is configured to permit a desired stretch. The rocking back stretcher comprises a cushion connected to a seat plate. The cushion is larger than the seat plate. A right corner brace and a left corner brace connected to the cushion. A right leg is connected to the right corner brace wherein the right leg has an oval shape. A left leg is connected to the left corner brace wherein the right leg has the oval shape. A front cross bar is connected to the right leg and the left leg. The front cross bar limits rotational movement of the left leg and the right leg forward. A rear cross bar is connected to the right leg and the left leg. The rear cross bar limits rotational movement of the left leg and the right leg rearward. Rocking forward or reward ends in the desired stretch.

In some embodiments, a right anti-skid strip is attached to the right leg to limit skidding. A left anti-skid strip is attached to the right leg to limit skidding. The front cross bar and the rear cross bar are connected to the right leg and the left leg with threaded cylinders to hold greater weight.

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of an embodiment of the invention shown in use.

FIG. 2 is a perspective view of an embodiment of the invention.

FIG. 3 is a side view of an embodiment of the invention.

FIG. 4 is a front view of an embodiment of the invention.

FIG. 5 is a top view of an embodiment of the invention.

FIG. 6 is a bottom view of an embodiment of the invention.

FIG. 7 is an exploded view of an embodiment of the invention.

FIG. 8 is a perspective view of the threaded cylinder.

### DETAILED DESCRIPTION OF CERTAIN EMBODIMENTS

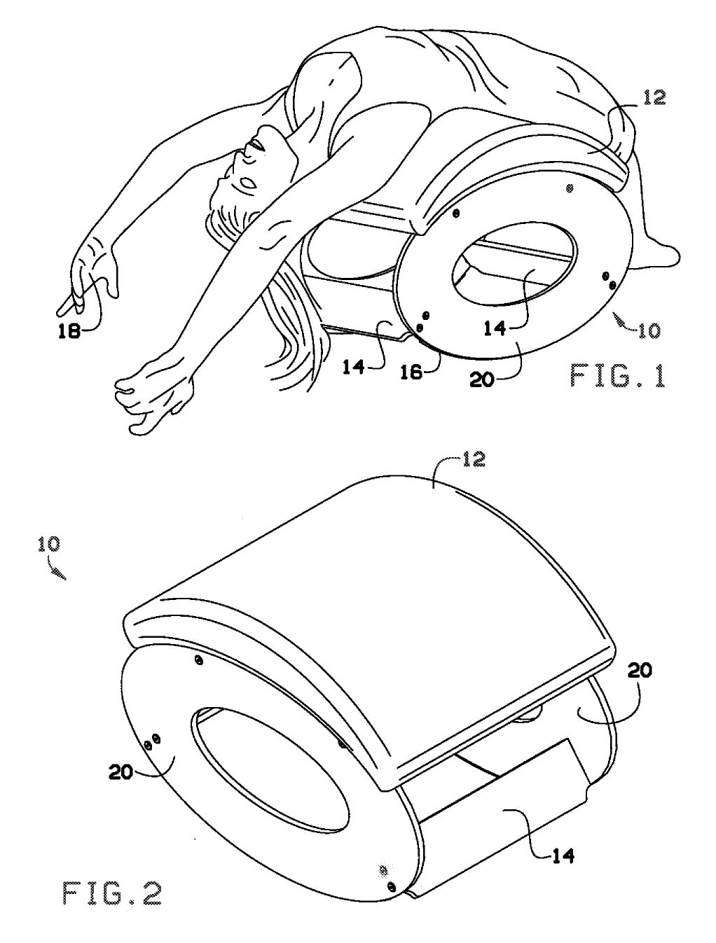

By way of example, and referring to FIG. 1, user 18 desires to exercise one's back or to sit on the same object. Rocking back stretcher 10 can be used to accomplish this. Rocking back stretcher 10 comprises right leg 20. Right leg 20 is mechanically coupled to front cross bar 14 with cross bar screws 30. Similarly, right leg 20 is mechanically coupled to rear cross bar 14 with cross bar screws 30. Front cross bar 14 limits rotational movement of the leg 20 and the right leg 20 forward.

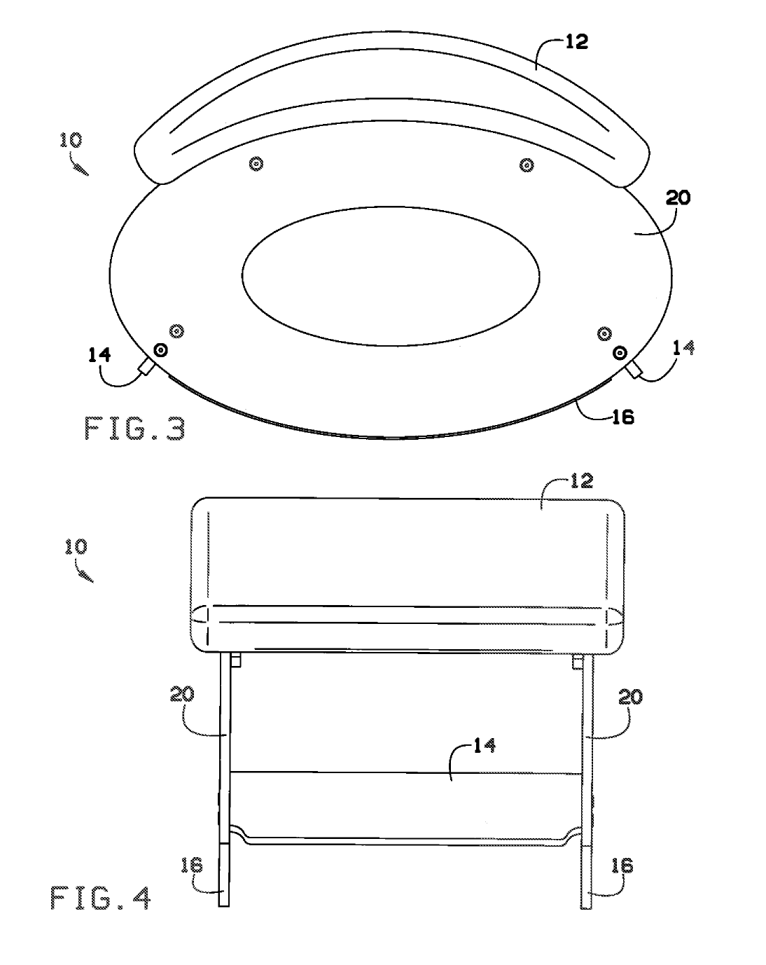

Turning to FIG. 2, FIG. 3 and FIG. 4, front cross bar 14 and rear cross bar 14 are connected to left leg 20 with cross bar screws 30. Front cross bar 14 limits rotational movement of left leg 20 and the right leg 20 forward. Rear cross bar 14 limits rotational movement of left leg 20 and the right leg 20 rearward. A portion of right leg 20 is covered by right anti-skid strip 16. A portion of left leg 20 is covered by left anti-skid strip 16.

In some embodiments, right leg 20 and left leg 20 have an oval shape. Right leg 20 and left leg 20 can be made from cabinet grade birch plywood. Likewise, rear cross bar 14 and front cross bar 14 can be made from cabinet grade birch plywood. In other embodiments, the wood parts of this device could potentially be made of materials other than wood, such as injection molded plastic, aluminum, and others.

In some embodiments, right anti-skid strip 16 and left anti-skid strip 16 can be made from rubber or any other material that can reduce the ability of right leg 20 and left leg 20 to roll. Right anti-skid strip 16 and left anti-skid strip 16 can be attached with an adhesive such as glue.

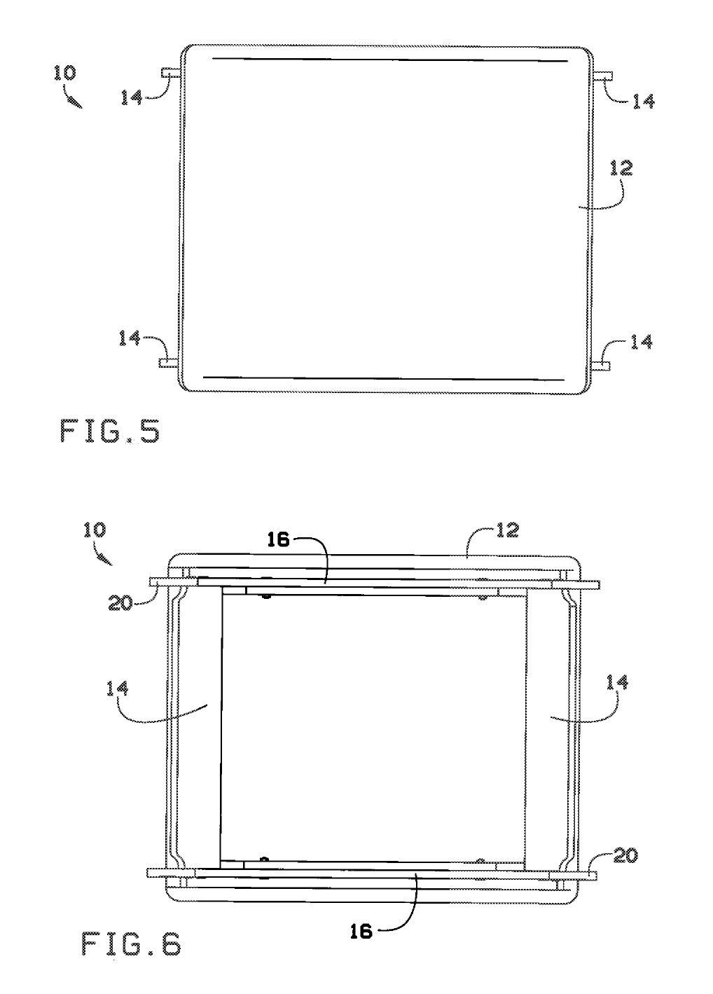

Turning to FIG. 5 and FIG. 6, right leg 20 is mechanically coupled to right corner brace 28 with fasteners 22 and threaded t-nuts 24. Likewise, left leg 20 is mechanically coupled to left corner brace 28 with fasteners 22 and threaded t-nuts 24. Seat plate 26 is mechanically coupled to right corner brace 28 and left corner brace 28 with fasteners 22. Cushion 12 is connected to seat plate 26. In an improvement over Palmisano, cushion 12 is wider than seat plate 26 which accommodates a wider user 18. Further, in some embodiments, cushion 12 is substantially wider than the distance between right leg 20 and left leg 20 providing greater comfort to a user sitting or lying on the surface depicted in Palmisano.

In some embodiments, seat plate 26 is a curved top consisting of ¼ inch plywood bender board. In some embodiments, cushion 12 comprises medium density foam, polyester batting and an anti-bacterial vinyl cover to provide comfort. In other embodiments, cushion 12 can utilize a leather cover. In some embodiments, the height of the highest point on cushion 12 to ground is twenty inches enabling a user to use rocking back stretcher 10 as a chair.

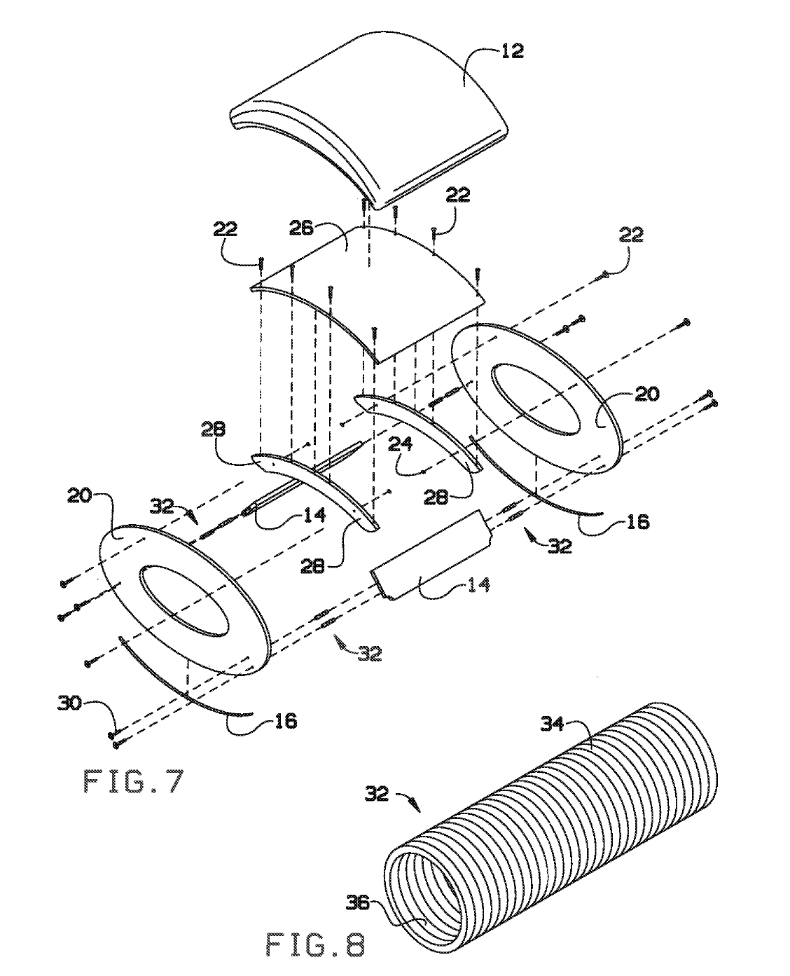

Turning to FIG. 7 and FIG. 8, in some embodiments, front cross bar 14 and rear cross bar 14 are perforated with threaded cavities. The threaded cavities can accommodate threaded cylinder 32. Threaded cylinder 32 comprises an inner surface covered with metal threads 34 and an outer surface comprising wood threads 34. A user can then insert cross bar screws 30 into threaded cylinder 32.

In some embodiments, threaded cylinder 32 is made of metal and cross bar screws 30 are metal screws. Threaded cylinders 32 provide a structure for holding more weight.

Returning to FIG. 1, to use rocking back stretcher 10, user 18 lies on cushion such that the user's back is against cushion 12. The user's legs can either be extended outward or bent at the knee (as shown) depending on the exercise. User 18 can rock back until rear cross bar 14 contacts the ground or until a desired stretch position is reached. Subsequently, user 18 can rock forward until front cross bar 14 contacts the ground or until a desired stretch position is reached. This process can continue as desired.

As a stretching device, user 18 can lay on cushion 12 top in a number of different ways to achieve a desired stretch. The height of rocking back stretcher 10, combined with the rocking ability, makes it much easier to achieve the desired results. Because the stretches are accomplished by placing the body in relaxed positions rather than using muscle exertion, the stretches are more complete and not strenuous. The rocking motion enables the user to focus the stretches to specific areas of the upper body.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A process for extending and stretching using a rocking back stretcher permitting a desired stretch; the process comprising:
lying on a cushion; wherein the cushion is connected to a seat plate; the seat plate is connected to a right corner brace and a left corner brace; the right corner brace is connected to a right leg; the left corner brace is connected to a left leg; the right leg and the left leg are connected to a front cross bar and a rear cross bar;
rocking forward until the desired stretch is achieved or the front cross bar makes contact with ground or rocking backward until the desired stretch is achieved or the rear cross bar makes contact with the ground; and
holding the desired stretch once it is achieved.

2. The process of claim 1, wherein the right leg and the left leg are connected to the front cross bar and the rear cross bar with threaded cylinders to accommodate more weight.

3. The process of claim 1, wherein the cushion comprises medium density foam, polyester batting and an anti-bacterial vinyl cover to provide comfort.

4. The process of claim 1, wherein the cushion is larger than the seat plate.

5. A rocking back stretcher configured to permit a desired stretch; the rocking back stretcher comprising:
a cushion connected to a seat plate; wherein the cushion is larger than the seat plate;
a right corner brace and a left corner brace connected to the cushion;
a right leg connected to the right corner brace wherein the right leg has an oval shape;
a left leg connected to the left corner brace wherein the right leg has the oval shape;
a front cross bar connected to the right leg and the left leg; wherein the front cross bar limits rotational movement of the left leg and the right leg forward;
a rear cross bar connected to the right leg and the left leg; wherein the rear cross bar limits rotational movement of the left leg and the right leg rearward;
wherein rocking forward or reward ends in the desired stretch.

6. The rocking back stretcher of claim 5, further comprising:
a right anti-skid strip attached to the right leg to limit skidding; and
a left anti-skid strip attached to the left leg to limit skidding.

7. The rocking back stretcher of claim 5, wherein the front cross bar and the rear cross bar are connected to the right leg and the left leg with threaded cylinders to hold greater weight.
